(19) United States
(12) Patent Application Publication
(10) Pub. No.: US 2021/0128831 A1
(43) Pub. Date: May 6, 2021

(54) SYSTEMS AND METHODS FOR DETERMINING PLACEMENT OF WEARABLE DRUG DELIVERY DEVICES

(71) Applicant: Insulet Corporation, Acton, MA (US)

(72) Inventors: Ashutosh ZADE, San Diego, CA (US); Joon Bok LEE, Acton, MA (US); Yibin ZHENG, Hartland, WI (US); Rangarajan NARAYANASWAMI, Weston, MA (US); David NAZZARO, Groveland, MA (US); Thomas METZMAKER, Harvard, MA (US)

(21) Appl. No.: 17/085,344

(22) Filed: Oct. 30, 2020

Related U.S. Application Data
(60) Provisional application No. 63/056,537, filed on Jul. 24, 2020.
(60) Provisional application No. 62/930,853, filed on Nov. 5, 2019.

(51) Int. Cl. A61M 5/172; A61M 5/142; G16H 20/13; G16H 20/17; G16H 40/67; G06N 20/00; G06N 5/04

(52) U.S. Cl. CPC A61M 5/172; A61M 2230/201; G16H 20/13; G16H 20/17; G16H 40/67; G06N 20/00; G06N 5/04; A61M 2205/52; A61M 2205/3327; A61M 2205/502; A61M 2230/63; A61M 2205/3584; A61M 2205/3592; A61M 2205/8212; A61M 5/14248

(57) ABSTRACT

A wearable drug delivery device and method for optimizing performance thereof are provided. A system may include a processor operable with memory, and a drug delivery device and sensor coupled to a user, the sensor operable to detect characteristics of the delivery device. A receiver operable on the processor receives an input signal from the sensor, the input signal representing the detected characteristics. A controller operable on the processor receives the input signal from the receiver, and retrieves, from memory, a baseline characteristics. The controller may determine a location of the delivery device and a tissue profile of the injection location based on a comparison between the detected characteristics and the baseline characteristics. The controller may further control or modify delivery of a liquid drug from the delivery device in response to the location of the delivery device.

### CROSS-REFERENCE TO RELATED APPLICATIONS

This application claims the benefit of the filing date of U.S. Provisional Application Ser. No. 62/930,853, filed Nov. 5, 2019 and U.S. Provisional Application Ser. No. 63/056,537, filed Jul. 24, 2020, the entire contents of which are incorporated herein by reference.

### TECHNICAL FIELD

Embodiments of the present disclosure generally relate to management of drug delivery devices. More particularly, embodiments of the present disclosure relate to systems and methods for determining placement of wearable drug delivery devices.

### BACKGROUND

“Artificial pancreas” systems are medication delivery systems that typically monitor a user's glucose levels, determine an appropriate level of insulin for the user based on the monitored glucose levels, and subsequently dispense insulin to the user. Insulin delivery devices may be worn in a variety of areas on the body, thus allowing for site rotation and placement based on user comfort/preference. Patch-type pumps in particular may be placed almost anywhere on the body because no external tubing is present. Site rotation of the insulin delivery device is often done to control infusion rate and reliability, as well as to prevent excessive buildup of scar tissue, which may cause diffusion and absorption issues.

A need therefore exists for systems and methods that increase performance of medication delivery systems by determining placement of wearable drug delivery devices.

### SUMMARY

In one approach of the disclosure, a computer-implemented method may include receiving, by an input signal receiver operable on a processor, an input signal from a sensor coupled to a user, wherein the input signal represents one or more characteristics, detected by the sensor, of a drug delivery device coupled to the user. The computer-implemented method may further include retrieving, from a memory, a plurality of baseline characteristics, and determining, by a controller operable on the processor, a location of the drug delivery device and a tissue profile of the location of the drug delivery device by comparing the one or more characteristics to the plurality of baseline characteristics. The computer-implemented method may further include controlling, by the controller, delivery of a liquid drug from the drug delivery device in response to the location of the drug delivery device and the tissue profile of the location of the drug delivery device.

In another approach of the disclosure, an article comprising a non-transitory computer-readable storage medium may include instructions that, when executed by a processor, may enable a wearable drug delivery system to receive, by an input signal receiver operable on the processor, a plurality of input signals from a sensor coupled to a user, wherein the plurality of input signals represents one or more characteristics, detected by the sensor, of a drug delivery device coupled to the user. The non-transitory computer-readable storage medium may further include instructions that, when executed by the processor, enable the wearable drug delivery system to retrieve, from a memory, a plurality of baseline characteristics, and to determine, by a controller operable on the processor, a location of the drug delivery device by comparing the one or more characteristics to the plurality of baseline characteristics. The non-transitory computer-readable storage medium may further include instructions that, when executed by the processor, enable the wearable drug delivery device to control, by the controller, delivery of a liquid drug from the drug delivery device in response to the location of the drug delivery device and the tissue profile of the location of the drug delivery device.

In another approach of the disclosure, a wearable drug delivery system may include a processor operable with a memory, a drug delivery device coupled to a user, and a sensor coupled to the user, the sensor operable to detect one or more characteristics of the drug delivery device. The wearable drug delivery system may further include an input signal receiver operable on the processor to receive an input signal from the sensor, the input signal representing the one or more characteristics, and a controller operable on the processor to receive the input signal from the input signal receiver, and retrieve, from the memory, a plurality of baseline characteristics. The controller may be further operable on the processor to determine a location of the drug delivery device on the user and a tissue profile of the location of the drug delivery device based on a comparison between the one or more characteristics and the plurality of baseline characteristics. The controller may be further operable to control delivery of a liquid drug from the drug delivery device in response to the location of the drug delivery device and the tissue profile of the location of the drug delivery device

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates an exemplary wearable medication delivery system according to embodiments of the present disclosure;

FIG. 2 illustrates a top perspective view of a drug delivery device depicted in FIG. 1 according to embodiments of the present disclosure;

FIG. 3 is a block diagram of a portable/local wireless device according to embodiments of the present disclosure; and

FIG. 4 illustrates a method according to embodiments of the present disclosure.

The drawings are not necessarily to scale. The drawings are merely representations, not intended to portray specific parameters of the disclosure. The drawings are intended to depict exemplary embodiments of the disclosure, and therefore are not be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where embodiments of the methods are shown. The systems, devices, and methods may be embodied in many different forms and are not to be construed as being limited to the embodiments set forth herein. Instead, these embodiments are provided so the disclosure will be thorough and complete, and will fully convey the scope of the systems, devices, and methods to those skilled in the art.

As noted above, rotation between different injection sites is advised to maintain infusion rates and absorption rates, and to reduce accumulation of fat cells, which results in lipohypertrophy and delayed insulin action. Delayed insulin action may cause a subsequent over-delivery of insulin, which can lead to hypoglycemia or occlusion. Moreover, tracking infusion/pod sites is an additional burden on patients.

Knowledge of a current infusion site has many benefits, including the ability to adapt the timing and drug dosage based on site location, monitoring site rotation for the patient in a consistent pattern, understanding which site is effective for the patient, alerting the patient on avoiding certain sites based on his/her blood glucose control, and alerting the patient for mismatch of physical activity in relation to device placement.

Various embodiments of the present disclosure include systems and methods for improving performance of wearable drug delivery systems by determining placement of a drug delivery device on a user, and then providing suggestions to the user for relocating the drug delivery device. In some embodiments, sensors such as accelerometers, gyroscopes, altimeters, etc., may be used to determine drug delivery device placement on the body based on typical body movements. Additionally, in some embodiments, strength of a communication signal (e.g., Bluetooth Low Energy (BLE), Bluetooth, RF, NFC, etc.) may be used to infer proximity of two therapeutic devices, such as the drug delivery device and a glucose monitor/sensor. Using one or more of these sensing technologies, the system may determine placement of devices, for example, given a previous sampling of data collected and analyzed over a period of time. In some embodiments, the user may be able to confirm or adjust this device location estimation on a mobile or handheld device, such as a smartphone, smart watch, etc. Automating drug delivery device and/or sensor placement on the body advantageously reduces the number of steps required by the user, while also increasing safety and effectiveness of the drug delivery device.

Various embodiments of the present disclosure may further determine an acceleration profile of the drug delivery device when a cannula of the drug delivery device is fired to determine whether the drug delivery device site includes fatty, muscular, and/or scar tissue. For example, the force it takes to insert the cannula will be different when the cannula enters fatty tissue vs. muscle tissue, resulting in different acceleration patterns registered at cannula insertion. If the same acceleration patterns are seen too often upon cannula insertion at a particular placement site over a number of placements, or if scar tissue is detected at the placement site, the mobile or handheld device may suggest alternative device placement.

Various embodiments of the present disclosure may further determine a next device location based upon historical information regarding required insulin sensitivity, as some sites may provide better absorption than others, and therefore may require less insulin to remain within an optimal blood glucose range. Additionally, during certain days of the week patient activity may be higher, which generally requires less insulin. The level of activity and site location may be taken together to modulate insulin delivery. In another example, timing of a bolus dose after a meal may be modified due to variation in insulin absorption rates in different locations on the body.

Various embodiments of the present dislcosure may further combine both drug delivery device location and site tissue composition to influence current and/or future insulin delivery. For example, assessment of the tissue at the delivery site (e.g., muscular, fatty or scar tissue) of the drug delivery device can be used to determine future insulin dose size and/or timing. If the insulin sensitivity is detected to be lower by the AID's feedback control logic, e.g., due to a less sensitive current pod site and/or due to scar tissue, the AID algorithm can determine this to be a pre-occlusion or pre-negative outcome condition and warn the user in an effort to avoid potential over-delivery of insulin.

Various embodiments of the present dislcosure may further determine drug delivery device location with the aid of preexisting device markers. For example, during activation of the drug delivery device, a Personal Diabetes Manager (PDM) may be aligned to the markers on the drug delivery device. The process of tilting and aligning the PDM to the drug delivery device markers can determine the drug delivery device site by running an algorithm on the PDM. This embodiment alleviates additional sensor and computational dependency on the drug delivery device; however, there is an extra step the user has to undertake during drug delivery device activation.

Various embodiments of the present disclosure may further determine whether the drug delivery device is being used properly (e.g., placed in regions where subcutaneous therapy is recommended) and/or being used consistently according to a recommended/prescribed dosing schedule. In some embodiments, recommendations may be made to the user through prompts, such as one or more alerts or instructions delivered to the user's mobile or handheld device. For example, alternative locations may be recommended to the user when the current location is less conducive to subcutaneous therapy, or when the user has used the same drug delivery device site too many consecutive times.

Various embodiments of the present disclosure may further recognize unique motion signatures generated by body and/or limb movements. In some embodiments, the movements may be tracked by examining a gravity vector variation coupled with rotation of a gyroscope of the drug delivery device. For example, wearing the drug delivery device on the thighs and walking will produce a unique motion signature compared to wearing the drug delivery device on the arms, back, abdomen, etc., and walking. The signature can be further differentiated between the left and right side of the user's body and/or the front and back of the user's body.

Various embodiments of the present disclosure may further include the use of machine learning classifiers trained to classify the accelerometer and gyroscope data to a corresponding site location. For example, site locations to be classified may include left arm, right arm, left thigh, right thigh, abdomen right, abdomen left, lower back left and lower back right, etc. Machine learning techniques for classification may include supervised and unsupervised learning as well as deep learning. Motion transition points (e.g., rest to motion, motion to rest, rest, etc.), and sequence descriptors, for example, Markov chains, can be used to improve the machine learning models.

Various embodiments of the present disclosure may further include power optimization techniques to minimize the battery power draw as well as the sensor processing needs. For example, if a site has been detected, the sensor processing algorithm can go into a sleep state with the option of waking up after expiration of a predetermined interval, as desired.

Various embodiments of the present disclosure may further include the creation of user feedback and site quality reports. For example, closed loop insulin delivery performance can be evaluated with respect to the insulin pump site, and feedback to the patient on preferred site can be provided. In some embodiments, feedback may include a site rotation map, which provides visual instructions to the patient for future placement of the drug delivery device. Meanwhile, site history maps can be generated to visually demonstrate past placement of the drug delivery device. This may be helpful for both the patient and the patient's caregiver (e.g., physician) in evaluating site use and damage. Furthermore, mismatch between device placement and excess physical movement of a corresponding limb may also be flagged to the patient with alternate site suggestions.

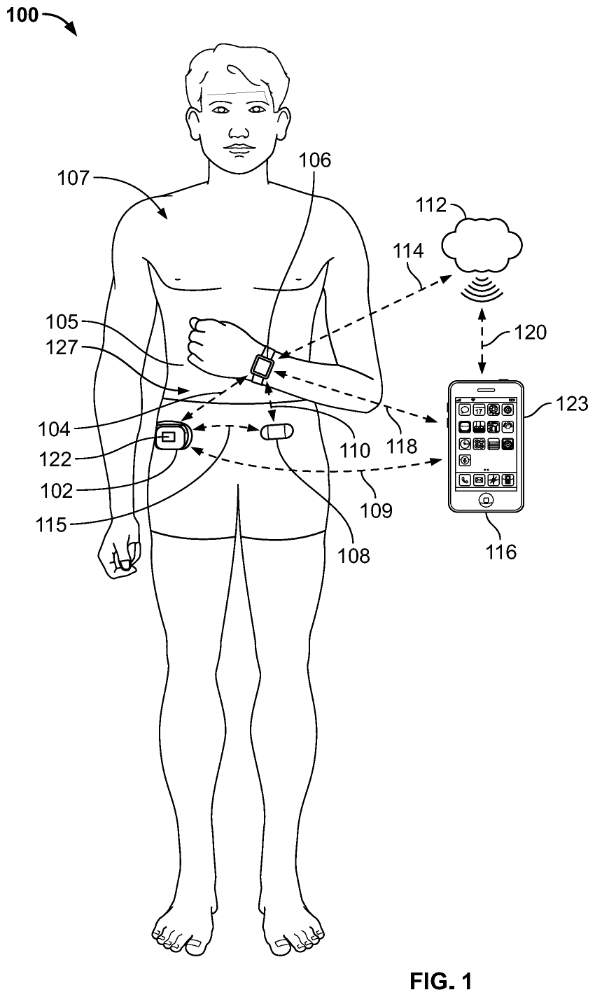

FIG. 1 illustrates a non-limiting example of a wearable medication delivery system (hereinafter “system”) 100. The system 100 may include a drug delivery device (hereinafter “device”) 102. As shown, the device 102 may be a wearable device, attached to a body 105 of a user 107 for delivering a medication (e.g., insulin) to the user 107. In some embodiments, a surface of the device 102 may include an adhesive to aid with attachment of the device 102 to the user 107.

The device 102 may include a number of components to facilitate delivery of a medication to the user. Although not shown, the device 102 may include a reservoir for storing the medication, a needle or cannula for delivering the medication into the body 105 of the user 107, and a pump for transferring the medication from the reservoir, through the needle or cannula, into the body 105 of the user 107. The device 102 may also include a power source, such as a battery, for supplying power to the pump and/or other components of the device 102. Although non-limiting, the device 102 may be the same or similar to an OmniPod® (Insulet Corporation, Acton, Mass.) insulin delivery device.

In some embodiments, the device 102 may also contain analog and/or digital circuitry for controlling the delivery of the medication. The circuitry may be implemented as a controller. The circuitry may include discrete, specialized logic and/or components, an application-specific integrated circuit, a microcontroller or processor that executes software instructions, firmware, or any combination thereof. In various embodiments, the control circuitry may be configured to cause the pump to deliver doses of the medication to the person at predetermined intervals. The size and/or timing of the doses may be programmed into the control circuitry using a wired or wireless link by the user 107 or by a third party, such as a health care provider.

Instructions for determining the delivery of the medication to the user (e.g., the size and/or timing of any doses of the medication) may originate locally (e.g., based on determinations made by the device 102) or may originate remotely, which are then provided to the device 102. Remote instructions may be provided to the device 102 over a wired or wireless link. The device 102 may execute any received instructions for the delivery of the medication to the user 107. In this way, under either scenario, the delivery of the medication to the user 107 may be automated.

In various embodiments, the device 102 may communicate via a wireless link 104 with an electronic device 106. The electronic device 106 may be any wearable wireless device such as, for example, a wearable computer (e.g., a smartwatch). The wireless link 104 may be any type of wireless link provided by any known wireless standard. As an example, the wireless link 104 may provide communications based on Bluetooth, Bluetooth Low Energy (BLE), Wi-Fi, radio frequency (RF), a near-field communication standard, a cellular standard, or any other wireless protocol.

Alternatively, or in addition thereto, the device 102 and/or the electronic device 106 may communicate with a portable/local wireless device 116. The local wireless device 116 may be a dedicated control or monitoring device (e.g., a Personal Diabetes Manager (PDM) and/or a custom handheld electronic computing device), mobile smartphone, laptop computer, tablet, desktop computer, or other similar electronic computing device. The local wireless device 116 may communicate with the device 102 via a wireless link 109, and may communicate with the electronic device 106 over a wireless link 118. The wireless links 109, 118 may be of the same type as the other wireless links described herein. A software application executing on the local wireless device 116 may be used to send commands to the device 102, e.g., either directly or via the electronic device 106, and to receive status/sensed information about the device 102.

Although not shown in detail for the sake of brevity, the local wireless device 116 may include various common computing elements, such as one or more processors, multi-core processors, co-processors, memory units, chipsets, controllers, peripherals, interfaces, oscillators, timing devices, video cards, audio cards, multimedia input/output I/O) components, power supplies, and so forth. The embodiments herein are not limited in this context.

As further shown, the system 100 may include a monitoring device or sensor 108, which may be worn on the body 105 of the user 107, or implanted within the user 107, and is used to collect information regarding one or more physical attributes or conditions of the person. In some embodiments, the sensor 108 may be a continuous glucose monitor (CGM). Although the sensor 108 is depicted as separate from the device 102, in various embodiments, the sensor 108 and the device 102 may be incorporated into the same unit. That is, in various other embodiments, the sensor 108 may be a part of the device 102 and contained within the same housing of the device 102.

In various embodiments, the sensor 108 may include one or more sensing elements, an electronic transmitter, receiver, and/or transceiver for communicating with the electronic device 106 over a link 110 or with device 102 over a link 115. The links 110, 115 may be the same type of wireless link as the links 104, 109, and 118 described above. Although not shown, the sensor 108 may also include a power source for supplying power to the sensing elements and/or transceiver. Communications provided by the sensor 108 may include data gathered from the sensing elements. This data may be transmitted continually, at periodic intervals, and/or during or after a change in sensed data (e.g., if a glucose level or rate of change in the level exceeds a threshold). The software application executing the algorithm may use this collected information to send a command to the device 102 to, for example, deliver a bolus to the user 107, change the amount or timing of future doses, or other commands. The sensor 108 may be any type of sensor and is not limited to a CGM. Furthermore, the sensor 108 may include multiple sensors housed in the same physical unit. Alternatively, the system may include multiple sensors 108 coupled to the user 107.

As further shown, the system 100 may include a location sensor 122 coupled to the user 107. The location sensor 122 may be operable to detect one or more characteristics of the device 102 and/or the body 105 of the user 107. In some embodiments, the location sensor 122 may be directly coupled to the body 105 of the user 107, for example, adjacent or near the device 102. In other embodiments, the location sensor 122 may be directly coupled to the device 102. In yet other embodiments, the location sensor 122 may be embedded or contained within the device 102. When the location sensor 122 is internal to the device 102, the location sensor 122 may be integrated with the analog and/or digital circuitry to be read directly by the embedded control software. The location sensor 122 may include one or more sensing elements, an electronic transmitter, receiver, and/or transceiver for communicating with the electronic device 106, the device 102, the sensor 108, and/or the local wireless device 116. The shared information may include handshake/pairing information, data, commands, status information, or any other such information. Embodiments herein are not limited in this context.

In various non-limiting embodiments, the location sensor 122 may be an accelerometer, a gyroscope, an altimeter, an inertial sensor (e.g., micro electromechanical systems (MEMS)), or the like. In one example, the location sensor 122 may be a 3-axis or tri-axial accelerometer capable of providing simultaneous measurements in three orthogonal directions (e.g., x, y and z). The magnitude of motion in each axial direction determines the motion in the particular direction. Alternatively, or additionally, the location sensor 122 may be any type of sensor capable of sensing one of more characteristics of the device 102 and/or the user 107 such as, for example, temperature, sound, infrared, pressure, radio signals, respiration, electrocardiogram (ECG) feedback, blood oxygen levels, heartbeat, audio, GPS locators, magnetic field, etc. Although only a single location sensor 122 is shown, the system 100 may include a greater or lesser number of sensors in communication with the other components of the system 100, e.g., the local wireless device 116. Embodiments herein are not limited in rhis context.

In some embodiments, the local wireless device 116 may include a controller (not shown) that executes instructions stored in a memory. In other embodiments, the controller may be part of the device 102. In either case, the controller may be operable to receive a signal from the location sensor 122 corresponding to the characteristics of the device 102 (e.g., acceleration, orientation, altitude, inertia, etc.) over a period of time, and compare the characteristics of the device 102 to one or more baseline characteristics, which may be stored in memory of the local wireless device 116 and/or the device 102. The baseline characteristics may be generated from previously detected characteristics of the user 107 at one or more device 102 locations, or from previously detected characteristics of a larger population of users for one more device locations. For exemple, in the case the user 107 has selected a torso or abdominal area 127 for current placement of the device 102, past data (e.g., previous 6 months, 12 months, etc.) regarding acceleration and/or inertia when the device 102 and the location sensor 122 are positioned along the abdominal area 127 may provide expected baseline acceleration/inertia characteristics for the abdominal area 127.

In other embodiments, the baseline characteristics may be more recent, for example, gathered as soon as the device 102 is activated and positioned on the user 107. In this case, a series of successive samples may be immediately taken to establish the baseline profile, e.g., from a 3-axis orientation. Reading of samples may be repeated at regular intervals following activation of the device 102, e.g., every hour, to build a history for the site location. In some cases, samples may be read during normal sleeping hours when user motion is limited, and tilt/orientation can further help narrow down the location of the device 102. A 3-axis profile may be generated to help establish one or more device sites, as each site will generate different profiles in each of the axes. For example, a device site in the back of the arm will have different motion profile (e.g., in steady state sitting, sleeping, etc.) vs motion profile on the hip. Additionally, using tilt and orientation detection, a side (e.g., left or right) of the body can also be determined. The site location for the device 102 may then be finalized and presented to the user 107 for confirmation.

In other examples, strength of signal or other characteristics of a signal between the device 102 and the sensor 108 may be used as another baseline characteristic. For example, a robust, consistent communication signal between the device 102 and the sensor 108 may infer an optimal proximity between the two. That is, a relatively weak detected signal may indicate that the device 102 and the sensor 108 are placed too far apart. Meanwhile, a distorted signal may indicate that the device 102 and the sensor 108 are placed too close together. For example, insulin in the sensor site may dampen the reading from the sensor 108, causing it to be inaccurate. In other embodiments, the sensor 108 may also include a location sensor (not shown), similar to the location sensor 122 of the device 102. Movement detected by the location sensor of the sensor 108 may be used to infer position of the sensor 108, which can then be compared to the position of the device 102 to determine a distance between the device 102 and the sensor 108. In some embodiments, the controller may retrieve the baseline characteristics from local memory (not shown) of the local wireless device 116 or from one or more remote memory sources.

Subsequent, real-time characteristics gathered from the location sensor 122 may be used to detect a deviation from the average or expected baseline characteristic(s), thus indicating whether the device 102 is currently in an optimal location on the body 105 of the user 107. Although non-limiting, an optimal location for the device 102 may mean an area of the body 105 where subcutaneous therapy is tolerated and most effective. Alternatively, or additionally, an optimal location for the device 102 may be an area of the body 105 where subcutaneous therapy is tolerated and most effective, yet not selected by the user 107 recently, e.g., within the past month, past three (3) months, etc. Varying, even moderately, the location of the device 102, may minimize excessive buildup of scar tissue.

The controller may then generate a feedback message, graphic, image, etc., to be displayed for the user 107, for example, via a display or GUI 123 of the local wireless device 116, indicating the location of the device 102. In response, the user 107 may be able to confirm or modify, via the GUI 123 or other input, the detected location of the device 102. In some embodiments, the user 107 may be required to provide a confirming input within a predetermined amount of time after the location feedback is provided. If the confirmation is not received within the predetermined amount of time, then the location of the device 102 may be assumed to be acceptable. For example, this may be the case when the deviation between the expected baseline characteristic(s) and the observed characteristic(s) is within an acceptable range. Alternatively, the location of the device 102 may be deemed unacceptable if the deviation between the expected baseline characteristic(s) and the observed characteristic(s) falls outside an acceptable range. In the case of the latter, delivery of the insulin to the user 107 may be blocked until the device 102 is relocated, or until the user 107 indicates that the current location is acceptable to him/her.

In some embodiments, the electronic device 106 and/or the device 102 may communicate with one more remote devices 112, which may include computers, servers, storage devices, cloud-based services, or other similar devices. The remote device 112 may be owned or operated by, for example, health-care companies or services, pharmacies, doctors, nurses, or other such medical entities. The remote device 112 may include a cloud-based data management system. The user 107 may wish, for example, to store data collected from the sensor 108 and/or the location sensor 122, store a record of device 102 locations, or back up other such information. As shown, the remote device 112 may communicate with the local wireless device 116 via a link 120.

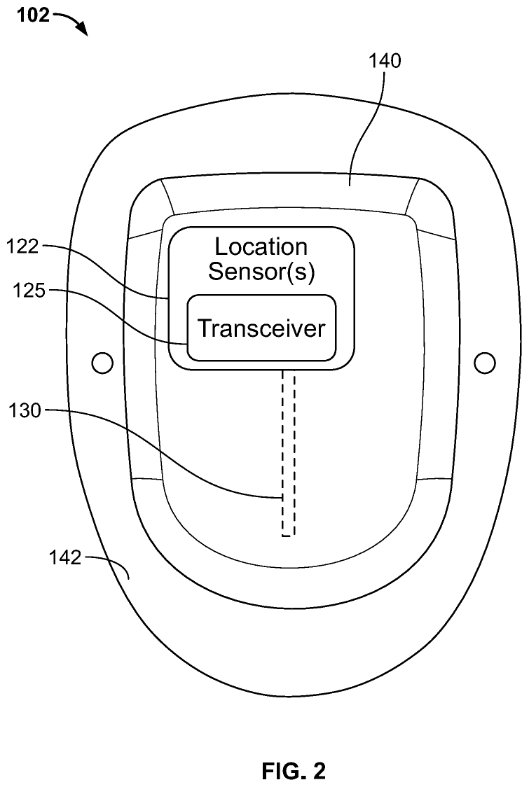

Turning now to FIG. 2 the device 102 according to embodiments of the present disclosure will be described in greater detail. As shown, the device 102 may include the location sensor 122 coupled thereto. In other embodiments, the location sensor 122 is contained within a housing 140 of the device 102. Further, the device 102 may include a pad 142 or other surface for adhering the device 102 to the user 107. The pad 142 may be coupled to a portion of the device 102, for example, an underside. The pad 142 may include an adhesive that may be used to attach the device 102 to the user. A needle or cannula 130 may be biased from the underside of the device 102 for insertion through the skin of the user 107, e.g., to deliver insulin or other liquid medication.

The location sensor 122 may include some or all the features described above. In various embodiments, the location sensor 122 may include a transceiver 125 to enable the location sensor and/or the device 102 to wirelessly communicate with any other device or component depicted in FIG. 1. The location sensor 122 may include at least one of an accelerometer, a gyroscope, a high-resolution altimeter, an inertial sensor, or the like. Furthermore, more than one type of location sensor 122 may be coupled to or embedded within the device 102.

In one embodiment, the location sensor 122 is an accelerometer which detects acceleration of the device 102 as the cannula 130 is inserted through a tissue of the user 107. Over a series of injections via the cannula 130, the controller may determine an acceleration profile for the device 102 to determine whether the injection site includes fatty, muscular, and/or scar tissue. For example, the force it takes to insert the cannula 130 will be different when the cannula 130 enters fatty tissue vs. muscle tissue, resulting in different acceleration patterns registered by the location sensor 122 during cannula 130 insertion. The acceleration patterns can be accumulated to develop a tissue profile or tissue composition for one or more injection sites. If the same acceleration patterns are seen, or if the acceleration patterns detect an unacceptable injection site, for example, due to a higher level of scar tissue, the local wireless device 116 may suggest alternative device placement for a subsequent therapy session and recommend to the user that this site not be used for a period of time, e.g., 3 months, 6 months etc., which may depend on the level of scar tissue as determined by the acceleration patterns. For example, a lower level of scar tissue may indicate the site is moderately healthy, in which case, the injection site may be revisited in the near future, as desired by the user.

Furthermore, in some embodiments, a change in acceleration for a given injection site over a period of time may indicate increasing amounts of scar tissue. For example, an acceleration profile that crosses a predetermined threshold may indicate that the injection site contains an unacceptable amount of scar tissue, likely from overuse. In some embodiments, an early warning may be provided to the user 107 so scar tissue can be avoided or at least minimized.

In some embodiments, the location sensor 122 includes both a 3-axis accelerometer and a 6-axis gyroscope at a known orientation within the device 102. The accelerometer and gyroscope may be processed at a selected frequency, wherein a sampled averaged accelerometer sensor points to the earth's gravity direction. The gravity vector components on the X, Y, Z axes of the accelerometer will vary with respect to the site location of the device 102 and body position of the user.

In some embodiments, the location sensor 122 may be removably attached to the device 102 so that the location sensor 122 may be used with a plurality of devices 102. Furthermore, the location sensor 122 may be sealed and waterproof. The location sensor 122 may have a battery, which is rechargeable using wired or wireless charging.

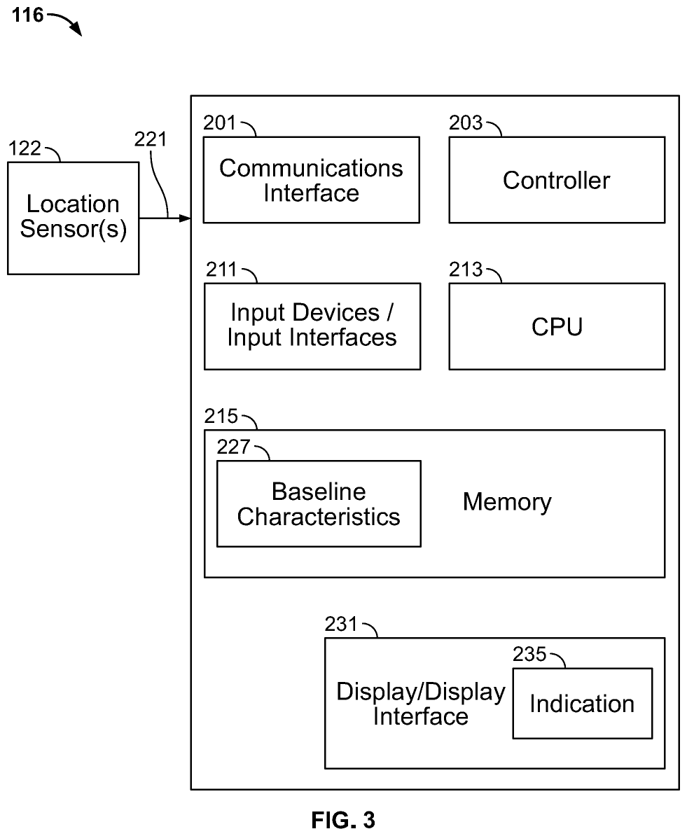

FIG. 3 illustrates an exemplary block diagram of the local wireless device 116. The local wireless device 116 may be, for example, a mobile phone, a smartphone, a laptop, a tablet, or any other handheld and/or portable electronic computing device. The local wireless device 116 may be the same or similar to the local wireless device 116 shown in FIG. 1 and described above. The local wireless device 116 may include a number of components, as shown. Specifically, the local wireless device 116 may include a communications interface 201 (e.g., an input signal receiver), a controller 203, input devices and input device interfaces 211, a central processing unit (CPU) or processor 213, and a memory 215.

The communications interface 201 may facilitate communication between the local wireless device 116 and a number of remote devices (not depicted). The communications interface 201 may provide communications over wired or wireless links or interfaces according to any known wired or wireless communication standard or protocol. For example, the communications interface 201 may enable the local wireless device 116 to communicate with one or more remote devices using, for example, Wi-Fi, a cellular communications standard, or Bluetooth.

The controller 203 may be a microcontroller or processor that executes software instructions, firmware, or any combination thereof. In some embodiments, the controller 203 executes instructions stored in memory 215. Specifically, the controller 203 may be operable on the CPU 213 to receive one or more input signals 221, via the communications interface 201, from the location sensor 122, the input signal 221 corresponding to the characteristics of the device 102 (FIGS. 1-2) detected by the location sensor 122. The controller 203 is operable on the CPU 213 to retrieve from memory 215 a plurality of baseline characteristics 227. In other embodiments, the baseline characteristics 227 are located remote from the local wireless device 116. The controller 203 is operable on the CPU 213 to compare the sensed characteristics of the location sensor 122 received through the input signal 221 to the baseline characteristics 227.

The baseline characteristics 227 may be an average of previously detected characteristics of the user 107 and/or device 102 at one or more device locations (e.g., arm, stomach, back, etc.), or from detected characteristics of a larger population of users. For example, the baseline characteristics 227 could incorporate aggregate data collected regarding favorite/common places for a group of similar users. In other examples, the baseline characteristics 227 may also incorporate other data, such as time of day, day of the week, whether the user is determined to be in a sleep state or awake, etc.

The controller 203 is operable on the CPU 213 to then display on a display/display interface 231 of the local wireless device 116, an indication 235 of the location of the device 102 on the user. The indication 235 may be a message, graphic, image, etc. The indication 235 may also provide instructions to the user on how or where to reposition the device 102 (e.g., “left arm has recently been used—move pod to torso”). In another example, the instructions may indicate to the user which locations are common/uncommon so the user can consider altering where the device 102 is positioned. In another example, the instructions may be provided during deactivation of the device 102. In yet another example, the local wireless device 116 may store the instructions and display to the user when a different device is subsequently activated and is ready to be positioned.

In some embodiments, the controller 203 is further operable on the CPU 213 to receive an input from the user regarding the placement of the device and/or the sensor 108. For example, the input from the user may be delivered through display/display interface 231 or the input devices/input device interface 211, the latter representing any number of input devices and interfaces that may process any inputs provided through an input device. For example, the input devices 211 may include a mouse, a keyboard, a touchscreen, and/or a microphone. The input device interfaces 211 may include one or more receivers for receiving input signals from any corresponding input device.

The CPU 213 may be a processor for executing instructions stored in the memory 215. The CPU 213 may control and direct operation of any of the components of the local wireless device 116. In particular, the CPU 213 may control the operation or functionality of the communications interface 201, the controller 203, and the input devices/input device interfaces 211.

The communications interface 201, the controller 203, and the input devices/input device interfaces 211 may be implemented in hardware, software, or any combination thereof. The local wireless device 116 may include other modules, components, or devices implemented in hardware, software, or any combination thereof and not shown in FIG. 3 to facilitate communication with remote devices, the receiving of input signals from a user, the receiving of the input signal 221 from the location sensor 122, and the presentation of visual information to the user via the display 231.

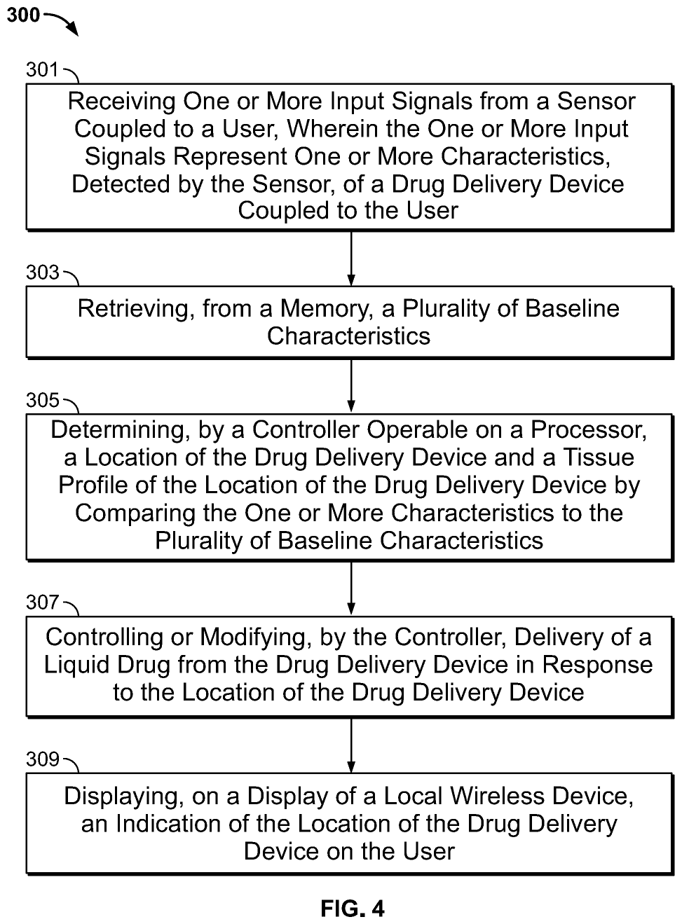

FIG. 4 illustrates a method 300 for determining placement of wearable drug delivery devices in accordance with the embodiments described herein. At block 301, the method 300 may include receiving one or more input signals from a sensor coupled to a user, wherein the one or more input signals represent one or more characteristics, detected by the sensor, of a drug delivery device coupled to the user. In some embodiments, the sensor may be an accelerometer, a gyrometer, a high-resolution altimeter, or an inertial sensor. In some embodiments, the accelerometer is a 3-axis or tri-axial accelerometer capable of providing simultaneous measurements in three orthogonal directions (e.g., x, y and z), wherein the magnitude of motion in each axial direction determines the motion in the particular direction. In some embodiments, more than one sensor may be coupled to the user. For example, the accelerometer may be combined with a 6-axis gyroscope. In some embodiments, the drug delivery device may include a pump for delivering insulin to the user. In some embodiments, the sensor may be directly coupled to, or integrated within, the drug delivery device.

In some embodiments, the one or more characteristics may be detected while the user is in a sleep state. Tilt/orientation samples detected during normal sleeping hours, when user motion is typically limited, can further help narrow down the location of the device 102. In some embodiments, the one or more characteristics may be detected during insertion of the cannula of the drug delivery device.

At block 303, the method 300 may include retrieving, from a memory, a plurality of baseline characteristics. In some embodiments, the baseline characteristics may be generated from previously detected characteristics of the user at one or more device locations, or from previously detected characteristics of a larger population of users for one or more device locations. In some embodiments, the memory may be locally or remotely located. Using any variety of sensing technologies, the controller may establish historical baseline characteristics of the drug delivery device after time (t), where t is a sampling window for data to be collected and analyzed by the controller.

More specifically, in some embodiments, the baseline characteristics may include tissue profiles or a tissue composition of tissue at one or more injection sites. In some embodiments, tissues profiles are generated from acceleration data for the drug delivery device collected when the cannula of the drug delivery device is fired. That is, the force it takes to insert the cannula will be different when the cannula enters fatty tissue vs. muscle tissue, resulting in different acceleration patterns registered during cannula insertion. For power savings, in some embodiments, at initial entry of the cannula the accelerometer will be sampled at a higher frequency as compared to the remainder of the time.

In some embodiments, the baseline characteristics may include historical information regarding required insulin sensitivity, as some sites may provide better sensititiy than others, i.e., may require less insulin to remain in range.

In some embodiments, the baseline characteristic may include date and/or time information for the user and/or a group of similar users. For example, user activity may be collected and then averaged for each day of the week. Some users may have lower activity levels during the week and higher activity levels on the weekend, for example. Furthermore, baseline characteristics may include data collected while the user is in a sleep state (e.g., either sleeping or in a reclined position for a period of time). This information can be used to guide insulin delivery requirements, as generally less insulin is required when users are more active.

In some embodiments, the baseline characteristics may include data collected during activation of the drug delivery device wherein a PDM may be aligned to markers on the drug delivery device. Acceleration of the device, which is recorded as the PDM is tilted/aligned to the device markers, can be aggregated to help determine site location.

In some embodiments, the baseline characteristics may include motion signatures generated by body/limb movements, which are tracked by examining the gravity vector variation coupled with the rotation of the gyroscope. For example, wearing the insulin pump on the thighs and walking will produce a unique motion signature compared to wearing the device on the arms/back/abdomen. Further, the signature can be used to further differentiate motion between the left, right, front, and/or back sides of the body. For example, when the device is worn on the arm, the device will have unique motion signatures during regular activities such as walking, eating, working, etc., which will differ between left and right arms. Furthermore, devices worn on the abdomen/back will have unique signatures during walking/postural changes, such as sitting, standing, bending etc.

In some embodiments, the baseline characteristics may continually evolve. For example, the controller may classify accelerometer and gyroscope data by including therein machine learning classifiers, which are trained to a corresponding site location for the device. For example, site location classifications may include left arm, right arm, left thigh, right thigh, abdomen right, abdomen left, lower back left and lower back right, etc. Machine learning techniques to provide this classification may include supervised learning, unsupervised learning, reinforcement learning, as well as deep learning (e.g., artificial neural networks). Over time, relevant aggregate information is retrieved and associated with one or more of the classifications.

At block 305, the method 300 may include determining, by a controller operable on a processor, a location of the drug delivery device and/or a tissue profile of tissue at the location of the drug delivery device by comparing the one or more characteristics to the plurality of baseline characteristics. In some embodiments, the location may be confirmed during typical sleep hours and/or when the user is detected to be in a sleep state. During this time, motion may be limited, and the tilt/orientation of the device can further help to identify the device location.

In some embodiments, the controller may determine whether a deviation between the one or more characteristics and the plurality of baseline characteristics is within an acceptable range. In some embodiments, the controller can infer device placement on the body based on typical body movements.

In some embodiments, the controller may determine a tissue profile or type at the location of the drug delivery device by comparing the one or more characteristics detected during the insertion of the cannula of the drug delivery device, such as acceleration, to the historical baseline acceleration data gathered during previous cannula injections.

In some embodiments, the controller may employ a machine learning model or algorithm to determine device location. For example, as noted above, the controller may determine a device location based on historical accelerometer and gyroscope data using machine learning. Although non-limiting, machine learning may include neural networks, regression algorithms, instance-based algorithms (e.g., k-Nearest Neighbor), decision-tree algorithms, Bayesian algorithms, clustering algorithms, association-rule-learning algorithms, deep-learning algorithms, dimensionality-reduction algorithms, ensemble algorithms, and any other suitable machine-learning algorithms. In some embodiments, the machine-learning models may be trained to each device site (e.g., left/right arm, back, thigh, etc.) using any suitable training algorithm, including supervised learning based on labeled/classified data, unsupervised learning based on unlabeled/unclassified data, and/or semi-supervised learning based on a mixture of labeled and unlabeled/classified data. In some embodiments, motion transition points (e.g., rest to motion, motion to rest, rest, etc.), and sequence descriptors, for example, Markov chains, can be used to improve the machine learning model.

At block 307, the method 300 may include controlling or modifying, by the controller, delivery of a liquid drug from the drug delivery device in response to the location of the drug delivery device. In some embodiments, delivery of the liquid drug may further be controlled based on a tissue profile of the location of the drug delivery device detected during the insertion of the cannula of the drug delivery device. In one embodiment, the controller may modify delivery timing of a bolus dose of the liquid drug. For example, the location of the device may influence how soon after a meal the bolus dose is delivered due to varying insulin absorption rates at different spots of the body. The stomach is generally the area of the body that provides the fastest absorption for most people, followed by the upper arms, thighs, and upper buttocks. Therefore, the bolus dose may be delayed following the meal for a longer period of time and/or delivered more slowly when the device is placed on the abdomen, and delivered relatively sooner and/or more rapidly when the device is placed on the upper buttocks, for example. In some embodiments, the bolus dose may be delayed by a predetermined time period established for each injection site. In some embodiments, meal detection may occur automatically based on a combination of inputs including, but not limited to, detected glucose levels by the CGM, body position of the user (e.g., seated position), time of day, etc. In other embodiments, the user may provide feedback to the device or the local wireless device indicating/confirming mealtime.

In some embodiments, the controller may control delivery of the liquid drug by modifying an infusion rate of the device based on the location of the device, wherein the infusion rate may refer to the size and/or timing of each delivery dose. For example, in those areas of the body with lower absorption rates, the infusion rate may be increased. Inversely, areas of the body with higher absorption rates may require a lower infusion rate. In some embodiments, the infusion rates will be modified on an individualized basis that is a function of the infusion site and individual variabilities of the site response to the uptake and transport of insulin. The infusion rate may also be modulated by the duration of the site usage. For example, increased age/usage of a particular site may decrease absorption and transport of the insulin.

At block 309, the method 300 may optionally include displaying, on a display of a local wireless device, an indication of the location of the drug delivery device on the user. The method may further include displaying to the user the type of tissue (e.g., muscle, fat, scar, and combinations thereof) at the injection site. In some embodiments, the controller may generate instructions displayable on a display, indicating to the user how to reposition the drug delivery device so the deviation between the one or more characteristics and the plurality of baseline characteristics is within the acceptable range. For example, a graphic or image may be provided to the user indicating or providing suggestions as how to reposition the drug delivery device to a more optimal position.

In some embodiments, the drug delivery device and/or the local wireless device may include one or more user output devices that may be used to provide an alarm, alert, or indication to the user that an instruction for drug delivery device placement has been determined or received. This indication may be audible, visual, and/or vibrational for example. In various embodiments, the indication may include one or more flashing light emitting diodes (LED) and/or a vibration provided by the local wireless device.

In some embodiments, after determining the location of the device on the body, a signal output of the device may be modified accordingly. For example, the LED may flash green in the case the device is located on an area of high absorption on the body (e.g., stomach) and flash yellow (e.g., when used on basal-only device) in the case the device is positioned on the lower back. In another example, volume of beeping or type of beeping from the device may be modified to indicate a desired feedback based on the location.

In some embodiments, the method 300 may optionally include receiving an input from the user, the input providing feedback regarding the location of the delivery device on the user. For example, the user may confirm or deny whether the location of the delivery device generated by the controller is correct. The input from the user may be received at any variety of input devices, such as a button, a touch screen, or an accelerometer (e.g., such that the input may be a tapping or movement of the local wireless device).

In some embodiments, the system may create one or more user feedback and site quality reports. For example, closed loop insulin delivery performance can be evaluated with respect to the device site, and feedback to the user and/or the user's physician on preferred site(s) will be provided. In some embodiments, feedback may include a site history map generated to visually demonstrate past placement of the device. For example, sequentially numbered graphics or icons may be superimposed over a body chart/picture and displayed to the user. The graphics or icons may vary in color, size, etc., and may provide additional information (e.g., infusion rate, dose schedule) when selected. Feedback may also include a future site rotation map, which provides visual instructions to the patient for future placement of the device. Furthermore, the report may highlight any mismatches between device placement, as well as any excess physical movement of a corresponding limb, which may warrant providing the user with alternate site suggestions.

In some embodiments, the method 300 may further include causing the sensor to enter a low-power or standby state after determining the location of the device. As the device is battery powered, minimizing the power draw as well as the sensor processing needs is beneficial. In one example, once a site has been detected, the sensor processing algorithm can go into a sleep state with the option of waking up, or transitioning back to a full-power state, at some predetermined future point in time. Alternatively, the sensor may be manually reactivated, either at the device or the local wireless device.

In sum, embodiments of the present disclosure provide an improved wearable drug delivery device system by increasing accuracy and knowledge of drug delivery device placement, which improves treatment effectiveness. Furthermore, unlike some prior approaches that attempt to track injection sites by taking pictures, an inexpensive sensor may be incorporated into the device to detect device and/or site position in an efficient manner without consuming significant computing resources or power.

Examples of a computer-readable storage medium or machine-readable storage medium may include any tangible media capable of storing electronic data, including volatile memory or non-volatile memory, removable or non-removable memory, erasable or non-erasable memory, writeable or re-writeable memory, and so forth. Examples of computer-executable instructions may include any suitable type of code, such as source code, compiled code, interpreted code, executable code, static code, dynamic code, object-oriented code, visual code, and the like. The storage medium may include instructions to be executed by the processor for implementing the user interfaces described herein. The embodiments are not limited in this context.

As used in this application, the terms “system” and “component” and “module” are intended to refer to a computer-related entity, either hardware, a combination of hardware and software, software, or software in execution, examples of which are provided by the exemplary computing architectures herein. For example, a component can be, but is not limited to being, a process running on a computer processor, a computer processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments also incorporating the recited features.

The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein.

The phrases “at least one”, “one or more”, and “and/or”, as used herein, are open-ended expressions, including conjunctive and disjunctive, in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure. The directional references do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer two elements are directly connected and in fixed relation to each other.

Still furthermore, although the illustrative method 300 is described above as a series of acts or events, the present disclosure is not limited by the illustrated ordering of such acts or events unless specifically stated. For example, some acts may occur in different orders and/or concurrently with other acts or events apart from those illustrated and/or described herein, in accordance with the disclosure. In addition, not all illustrated acts or events may be necessary to implement a methodology in accordance with the present disclosure. Furthermore, the method 300 may be implemented in association with the formation and/or processing of structures illustrated and described herein as well as in association with other structures not illustrated.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, the present disclosure has been described herein in the context of a particular implementation in a particular environment for a particular purpose. Those of ordinary skill in the art will recognize the usefulness is not limited thereto and the present disclosure may be beneficially implemented in any number of environments for any number of purposes. Thus, the claims set forth below are to be construed in view of the full breadth and spirit of the present disclosure as described herein.

What is claimed is:

1. A computer-implemented method, comprising:
receiving, by an input signal receiver operable on a processor, an input signal from a sensor coupled to a user, wherein the input signal represents one or more characteristics, detected by the sensor, of a drug delivery device coupled to the user;
retrieving, from a memory, a plurality of baseline characteristics;
determining, by a controller operable on the processor, a location of the drug delivery device by comparing the one or more characteristics to the plurality of baseline characteristics; and
controlling or modifying, by the controller, delivery of a liquid drug from the drug delivery device in response to determining the location of the drug delivery device.

2. The computer-implemented method of claim 1, further comprising integrating the sensor within the drug delivery device.

3. The computer-implemented method of claim 1, further comprising detecting, by the sensor, the one or more characteristics while the user is in a sleep state.

4. The computer-implemented method of claim 1, further comprising:
detecting, by the sensor, the one or more characteristics during an insertion of a cannula of the drug delivery device; and
determining, by the controller, a tissue profile of the location of the drug delivery device based on the one or more characteristics detected during the insertion of the cannula of the drug delivery device.

5. The computer-implemented method of claim 1, further comprising:
displaying, on an interface of a local wireless device, an indication of the location of the drug delivery device on the user;
determining, by the controller, a deviation between the one or more characteristics and the plurality of baseline characteristics is outside an acceptable range; and
generating, by the controller, one or more instructions displayable on the interface, the one or more instructions indicating to the user how to reposition the drug delivery device to bring the deviation between the one or more characteristics and the plurality of baseline characteristics within the acceptable range.

6. The computer-implemented method of claim 5, further comprising receiving a user input via the interface of the local wireless device, the user input providing feedback regarding the location of the drug delivery device on the user.

7. The computer-implemented method of claim 1, wherein controlling delivery of the liquid drug from the drug delivery device comprises modifying delivery timing of a bolus dose.

8. The computer-implemented method of claim 1, wherein controlling delivery of the liquid drug from the drug delivery device comprises modifying an infusion rate of the liquid drug based on the location of the drug delivery device.

9. The computer-implemented method of claim 8, wherein determining the location of the drug delivery device comprises classifying acceleration data received by the sensor with machine learning classifiers.

10. The computer-implemented method of claim 9, further comprising causing the sensor to enter a low-power state after determining the location of the drug delivery device.

11. The computer-implemented method of claim 10, further comprising causing the sensor to enter a full-power state from the low-power state after a predetermined period of time.

12. An article comprising a non-transitory computer-readable storage medium including instructions that, when executed by a processor, enable a wearable drug delivery system to:
receive, by an input signal receiver operable on the processor, a plurality of input signals from a sensor coupled to a user, wherein the plurality of input signals represents one or more characteristics, detected by the sensor, of a drug delivery device coupled to the user;
retrieve, from a memory, a plurality of baseline characteristics;
determine, by a controller operable on the processor, a location of the drug delivery device by comparing the one or more characteristics to the plurality of baseline characteristics; and
control or modify, by the controller, delivery of a liquid drug from the drug delivery device in response to determining the location of the drug delivery device.

13. The article of claim 12, the non-transitory computer-readable storage medium further including instructions that, when executed by the processor, enable the wearable drug delivery system to:
determine, by the controller operable on the processor, a deviation between the one or more characteristics and the plurality of baseline characteristics is outside an acceptable range; and
generate, by the controller operable on the processor, instructions displayable on a display, the instructions indicating to the user how to reposition the drug delivery device so the deviation between the one or more characteristics and the plurality of baseline characteristics is within the acceptable range.

14. The article of claim 13, the non-transitory computer-readable storage medium further including instructions that, when executed by the processor, enable the wearable drug delivery system to:
detect, by the sensor, the one or more characteristics during an insertion of a cannula of the drug delivery device into the user; and
determine, by the controller, a tissue profile of a tissue at the location of the drug delivery based on an acceleration of the drug delivery device detected by the sensor during the insertion of the cannula of the drug delivery device into the tissue.

15. The article of claim 13, the non-transitory computer-readable storage medium further including instructions that, when executed by the processor, enable the wearable drug delivery system to receive a user input via an interface of a local wireless device, the user input providing feedback regarding the location of the drug delivery device on the user.

16. The article of claim 12, the non-transitory computer-readable storage medium further including instructions that, when executed by the processor, enable the wearable drug delivery system to control delivery of the liquid drug from the drug delivery device by modifying delivery timing of a bolus dose.

17. The article of claim 16, the non-transitory computer-readable storage medium further including instructions that, when executed by the processor, enable the wearable drug delivery system to control delivery of the liquid drug from the drug delivery device by delaying delivery of the bolus dose.

18. The article of claim 12, the non-transitory computer-readable storage medium further including instructions that, when executed by the processor, enable the wearable drug delivery system to modify an infusion rate of the liquid drug based on the location of the drug delivery device.

19. The article of claim 18, the non-transitory computer-readable storage medium further including instructions that, when executed by the processor, enable the wearable drug delivery system to classify acceleration data received by the sensor with machine learning classifiers.

20. The article of claim 12, the non-transitory computer-readable storage medium further including instructions that, when executed by the processor, enable the wearable drug delivery system to:
cause the sensor to enter a low-power state after determining the location of the drug delivery device; and
cause the sensor to enter a full-power state from the low-power state after a predetermined period of time.

21. A wearable drug delivery system, comprising:
a processor operable with a memory;
a drug delivery device coupled to a user;
a sensor coupled to the user, the sensor operable to detect one or more characteristics of the drug delivery device;
an input signal receiver operable on the processor to receive an input signal from the sensor, the input signal representing the one or more characteristics; and
a controller operable on the processor to:
receive the input signal from the input signal receiver;
retrieve, from the memory, a plurality of baseline characteristics;
determine a location of the drug delivery device on the user based on a comparison between the one or more characteristics and the plurality of baseline characteristics; and
control delivery of a liquid drug from the drug delivery device in response to the location of the drug delivery device.

22. The wearable drug delivery system of claim 21, wherein the sensor is directly coupled to the drug delivery device, and wherein the sensor is further operable to detect the one or more characteristics while the user is determined to be in a sleep state.

23. The wearable drug delivery system of claim 21, wherein the sensor is further operable to detect the one or more characteristics during an insertion of a cannula of the drug delivery device, and wherein the controller is further operable on the processor to determine a tissue profile of the location of the drug delivery device based on the one or more characteristics detected during the insertion of the cannula of the drug delivery device.

24. The wearable drug delivery system of claim 21, further comprising a local wireless device, wherein the controller is further operable on the processor to display on an interface of the local wireless device an indication of the location of the drug delivery device on the user, and wherein the controller is further operable on the processor to receive an input from the user, via the interface of the local wireless device, regarding the location of the drug delivery device on the user.

25. The wearable drug delivery system of claim 21, wherein the local wireless device is a mobile smart device, and wherein the sensor is an accelerometer, a gyrometer, a high-resolution altimeter, or an inertial sensor.

26. The wearable drug delivery system of claim 21, wherein the controller is further operable on the processor to control delivery of the liquid drug from the drug delivery device by modifying delivery timing of a bolus dose.

27. The wearable drug delivery system of claim 21, wherein the controller is further operable on the processor to control delivery of the liquid drug from the drug delivery device by modifying an infusion rate of the liquid drug based on the location of the drug delivery device.

28. The wearable drug delivery system of claim 27, wherein the controller is further operable on the processor to determine the location of the drug delivery device by classifying acceleration data received by the sensor with machine learning classifiers.

29. The wearable drug delivery system of claim 21, further comprising causing the sensor to enter a low-power state after determining the location of the drug delivery device.
